(19) United States
(12) Patent Application Publication
(10) Pub. No.: US 2022/0233411 A1
(43) Pub. Date: Jul. 28, 2022

(54) RECONSTITUTABLE SANITISER

(71) Applicant: Godrej Consumer Products Ltd., Mumbai (IN)

(72) Inventors: Sonia SAMNANI, Mumbai (IN); Sandeep NAIK, Mumbai (IN); Venkateswara YADLAPALLI, Mumbai (IN)

(21) Appl. No.: 17/465,978

(22) Filed: Sep. 3, 2021

(30) Foreign Application Priority Data
Jan. 23, 2021 (IN) 202121003282

(51) Int. Cl. A61K 8/02; A61K 8/41; A61Q 17/00

(52) U.S. Cl. CPC A61K 8/0225; A61K 8/416; A61K 2800/882; A61K 2800/31; A61Q 17/005

(57) ABSTRACT

A reconstitutable sanitiser formulation which is non-alcoholic in nature and achieves up to 99.9% reduction in microbial load and provides long-lasting protection against microbes for 2-6 hrs.

### FIELD OF INVENTION

The present invention is in field of sanitiser for personal hygiene. More particularly, the present invention relates to reconstitutable sanitiser formulation for personal hygiene.

### BACKGROUND OF INVENTION

Most of sanitisers, currently available in market, contain alcohol, in form of ethanol or isopropanol in concentrations of above 60%. This high concentration of alcohol is very effective in killing most of the bacteria and deactivating most of viruses.

However, presence of alcohol possesses issues of its own. Firstly, alcohols are hygroscopic in nature, as result of which they readily absorbs moisture from skin, leaving the skin dry and sometimes itchy. Secondly, alcohols, by nature, are inflammable, as result of which they need to carefully handled, especially while working around flames, for instance while cooking or while performing rituals.

Furthermore, alcohols evaporate instantly, thereby instantly giving microbial protection, as microbes can re-colonise the skin surface once the alcohol evaporates, which is in mere minutes.

Therefore, there was a need to develop non-alcoholic sanitisers, which are easier to handle. With this view sanitisers containing non-flammable antimicrobial agent were developed. For example, the sanitiser composition claimed in U.S. Pat. No. 6,087,400, WO 2002/102151 and U.S. Pat. No. 9,775,346 use quaternary ammonium compound, such as, benzethonium chloride or benzalkonium chloride, as an antimicrobial agent. The use of antimicrobial agent eliminates need for using alcohol and also, are comparatively safe for handling.

Further, all the sanitisers, alcoholic or non-alcoholic, known in art are generally supplied as solutions or as gels. Being in liquid or semi-liquid form, these sanitisers are prone to spillage either during transportation or during usage.

Therefore, it is an object of the present invention to propose a sanitiser formulation which does not damage the skin and affords a long-lasting protection against microbes.

Another object of the invention is to provide a sanitiser formulation which is easy and economical to transport.

### SUMMARY OF INVENTION

Accordingly, in an aspect the invention provides a sanitiser formulation which contains a non-alcoholic microbicidal agent and moisturisers.

In further aspect the invention provides a sanitiser formulation in form of reconstitutable powder.

### DETAILED DESCRIPTION OF INVENTION

The present invention describes a reconstitutable sanitiser formulation in anhydrous powder form. Said powder form is easy to transport and is devoid of any alcoholic microbicide. The sanitiser of the invention is further provided with moisturizers to maintain the moisture of the skin.

For the purpose of this invention, the terms “a”, “an” and “the” encompass both singular and plural reference.

For the purpose of this invention, the term “microbe” and its variations thereof encompass both bacteria, protozoa and viruses which may be lingering on the surface of skin. The term, further, encompasses, pathogenic and non-pathogenic microorganisms and viruses.

In an embodiment, the reconstitutable sanitiser formulation of the invention may comprises of a antimicrobicidal agent a humectant a conditioning agent/Film former/Antistatic Agent a sequestering agent perfume, colour and Extract

Preferably, the antimicrobicidal agent is a Quaternary ammonium compounds, selected from the group consisting of mixture of alkyl dimethyl benzylammonium chlorides, generally grouped as benzalkonium chloride, dimethyl dodecyl ammonium chloride or bromide, cetyltrimethylammonium bromide, cetylpyridinium chloride etc or lactic Acid/natural actives. Therefore, the invention uses Cationic antimicrobial agent to achieve almost complete sanitisation of hands, killing up to 99.9% of microbial load. Further, as Cationic antimicrobial agent does not evaporate like alcohol, hence, the sanitiser of the invention provides a long-lasting microbicidal protection of 2-6 hrs with conditioning property

The humectant as used herein is selected from triethylene glycol, tripropylene glycol, propylene glycol, glycerine, sorbitol, hexylene and butylene glycol, urea, sucrose, collagen or milk protein as skin conditioning agent. The humectant retains the moisture of the skin, thereby preventing drying of the skin.

The conditioning agent/Film former/Antistatic Agent is selected from agent known in the prior arts. Preferably, the agent is selected from Polyquaternium compound, such as, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, Acrylamidopropyltrimonium Chloride/Acrylamide Copolymer, Gum or combinations thereof.

The conditioning agent locks the moisture of skin, thereby preventing the dryness and/or itchiness of skin caused due to alcohols present in sanitisers currently available in market.

The sanitiser further comprises of suitable sequestering agent, selected from EDTA and it salts thereof, citric acid, cyclodextrin, Sodium Gluconate, etc and the like.

Depending upon the end use for which the reconstitutable sanitiser of this invention is intended, it may further contain components such as perfumes, gums, flavouring agents, colouring agents, and inorganic or organic salts.

The sanitiser of the invention is in form of a powder which needs to be reconstituted before the use. As the sanitiser is formulated in powder form, it is easy and economical to transport as there is no spilling hazard involved.

Further, since the powder could be reconstituted, the sanitiser can be commercialised as such, in pouch form, this reduces the usage of plastic containers which are required for containing the liquid. This reduces plastic usage, as a single container can be used multiple times by emptying the pouch containing powder sanitizer composition into the container. The container may be an empty bottle, an empty bottle with a Flip top cap, or an empty pump spray bottle.

In an embodiment, when the user intends to use the sanitiser, following procedure needs to be followed:

Step a: Emptying the sachet containing 0.8 gm to 3 gm of reconstitutable powder adding sufficient quantity of 80 to 100 ml potable water;

Step b: adding the reconstitutable powder in the Potable water, and

Step c: shaking the mixture vigorously.

Selection/screening of correct conditioning/film forming/antistatic agent which get easily soluble within few mins to an hour and which is less hygroscopic or completely non hygroscopic in nature.

Select antimicrobial agent which is compatible with the other ingredients and soluble easily.

Screening of correct other ingredients which are compatible with each other.

In an advantageous embodiment, the sanitiser of the invention provides a long-lasting protection against microbes. As, the active Cationic antimicrobial ingredient of current invention stays on the skin surface for longer period of time as compared to alcohol.

In another advantageous embodiment, the sanitiser of the invention provides reduction of up to 99.9% microbial load for a 2-6 hrs.

In another advantageous embodiment, the presence of humectants and conditioning agents maintains the moisture in the skin thereby ensuring minimal damage to skin surface.

### EXAMPLES

The following sanitiser formulation was/were prepared in accordance with the invention. For use as a sanitising solution, the solid formulations was/were dissolved in water just prior to use.

### Example 1

| Sr. No. | Ingredients | Range (in %) |
|---|---|---|
| 1. | Humectant | 0.05-50.00 |
| 2. | Conditioning Agent | 1-90% |
| 3. | Cationic antimicrobial active | 0.001-20.00 |
| 4. | Preservative | 0.0-10.0 |
| 5. | Chelating agent | 1.66-10.00 |
| 6. | Colour | 0.0-5.00 |
| 7. | Perfume | 1.66-20.0 |
| 8. | Extracts | 0.0-5.0 |
| | TOTAL | 100.00 |

Process for preparation:
a) Mixing chelating agent, perfume & or colour, preservative;
b) Mixing conditioning agent, anti-microbial agent, humectant & or Extract and,
c) mixing of mixture of steps a and b uniformly.

Advantages:
1) The said formulation has the efficacy to kill the organism within seconds.
2) Formulation has a kill efficacy of 2-6 hrs as per residual efficacy study as per lab test report in the controlled environment.

We claim:

1. A reconstitutable personal hygiene product which forms a stable liquid upon reconstitution, wherein said personal hygiene product comprises:
a) a conditioning agent in a range of 1.0-90.0%;
b) a cationic antimicrobial active in range of 0.001 to 20%, and
c) a humectant in range of 0.05-50.0%

2. The reconstitutable personal hygiene product as claimed in claim 1, further comprising a perfume in range of 0.2-20.0%, colouring agent in range of 0.0-5.0%, a chelating Agent in range of 0.3-10.0%, an extract in range of 0.0-5.0%, or a combination thereof.

3. The reconstitutable personal hygiene product as claimed in claim 1, wherein the conditioning agent is selected from the group consisting of dry powder natural extracts of aloe vera, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, and Polyquaternium-47, Acrylamidopropyltrimonium Chloride, Acrylamide Copolymer, Gum, and combinations thereof.

4. The reconstitutable personal hygiene product as claimed in claim 1, wherein the Cationic antimicrobial active is selected from the group consisting of benzalkonium chloride, Benzylthonium chloride, dimethyl dodecyl ammonium chloride, dimethyl dodecyl ammonium bromide, cetyltrimethylammonium bromide, cetylpyridinium chloride, and combinations thereof.

5. The reconstitutable personal hygiene product as claimed in claim 2, wherein the chelating agent is selected from the group consisting of EDTA, Disodium EDTA, Sodium Gluconate, and combinations thereof.

6. The reconstitutable personal hygiene product as claimed in claim 1, wherein the product further comprises a humectant selected from the group consisting of sucrose, milk protein, and combinations thereof.

7. The reconstitutable personal hygiene product as claimed in claim 2, wherein the extract is selected from the group consisting of extracts of Aloe Vera, Neem Extract, Lemon, Rosemary, Chamomile, Tulsi, Apple, marigold, and combinations thereof.

8. The reconstitutable personal hygiene product as claimed in claim 1, wherein the product forms a stable liquid when mixed with water.

9. The reconstitutable personal hygiene product as claimed in claim 1 wherein the pH of the product is in range of 3.5-8.5.

10. The reconstitutable personal hygiene product as claimed in claim 1, wherein the viscosity of the product is in a range of 100 cps to 8000 cps.

11. A kit for cleansing comprising:
a. a sachet containing the reconstitutable personal hygiene product as claimed in claim 1, wherein the reconstitutable personal hygiene product is a reconstitutable powder, and
b. a container for reconstituting the reconstitutable product and dispensing the reconstituted product.

12. A kit for cleansing comprising:
a. a sachet containing the reconstitutable personal hygiene product as claimed in claim 3, wherein the reconstitutable personal hygiene product is a reconstitutable powder, and
b. a container for reconstituting the reconstitutable product and dispensing the reconstituted product.

13. A kit for cleansing comprising:
a. a sachet containing the reconstitutable personal hygiene product as claimed in claim 4, wherein the reconstitutable personal hygiene product is a reconstitutable powder, and
b. a container for reconstituting the reconstitutable product and dispensing the reconstituted product.

14. A kit for cleansing comprising:
a. a sachet containing the reconstitutable personal hygiene product as claimed in claim 10, wherein the reconstitutable personal hygiene product is a reconstitutable powder, and
b. a container for reconstituting the reconstitutable product and dispensing the reconstituted product.

15. The kit as claimed in claim 11, wherein the sachet contains 0.5 to 3 g of the reconstitutable powder.

16. A method for reconstituting the reconstitutable personal hygiene product as claimed in claim 1, comprising:
a. filling an empty container with water to produce a filled container;
b. emptying a sachet containing the reconstitutable personal hygiene product as claimed in claim 1 into the filled container, wherein the reconstitutable personal hygiene product is a reconstitutable powder;
c. shaking the filled container in order to disperse the reconstitutable powder, and
d. allowing the bottle to stand to obtain a stable viscous liquid, spray, or gel.

17. The method as claimed in claim 16, wherein the empty container is an empty bottle, an empty bottle with a Flip top cap, or an empty pump spray bottle.
